(19) United States
(12) Patent Application Publication
(10) Pub. No.: US 2010/0041969 A1
(43) Pub. Date: Feb. 18, 2010

(54) MEASURING DEVICE AND METHOD FOR OPTICALLY DETERMINING THE CONCENTRATION OF BLOOD SUGAR AND/OR LACTATE IN BIOLOGICAL SYSTEMS

(76) Inventor: Reinhard D. Beise, Ottenhofen (DE)

Correspondence Address: COLLARD & ROE, P.C., 1077 NORTHERN BOULEVARD, ROSLYN, NY, 11576, US

(21) Appl. No.: 12/450,189

(22) PCT Filed: Mar. 16, 2008
(86) PCT No.: PCT/DE2008/000438

§ 371 Date: Sep. 15, 2009

(30) Foreign Application Priority Data
Mar. 16, 2007 (DE) 10 2007 013 274.5
Jul. 12, 2007 (DE) 10 2007 032 849.6

(51) Int. Cl. A61B 5/1455; G01N 21/35

(52) U.S. Cl. 600/316

(57) ABSTRACT

The invention relates to a measuring device for optically determining the concentration of blood sugar and/or lactate in biological systems, comprising at least one IR radiation source, that radiates IR light on a volume that is to examined, and at least one measuring detector that detects light coming from the volume that is to be examined in order to determine the concentration of blood sugar and/or lactate, also by laymen in a simple manner and anywhere. According to the invention, the IR light radiated on the volume that is to be examined is supplied, prior to entry into the volume, to a reference detector.

The invention relates to a measurement device for optical determination of the concentration of blood sugar and/or lactate in biological systems, having at least one IR (infrared) radiation source that radiates IR light onto a volume to be investigated, having at least one measurement detector that absorbs light proceeding from the volume to be investigated, and having at least one reference detector to which the IR light radiated onto the volume to be investigated is passed before entry into the volume, whereby the radiation source, the reference detector, and the measurement detector are connected with one another by means of a lock-in, and an optical measurement path is formed between the IR radiation source and the measurement detector, having a first measurement path characteristic, and an optical reference path is formed between the IR radiation source and the reference detector, having a second measurement path characteristic that deviates from the first measurement path characteristic. Also, the invention relates to a method for optical determination of the concentration of blood sugar and/or lactate in biological systems, in which IR light is radiated onto a volume to be investigated, along an optical measurement path, and a value relevant to the concentration is determined from the light that proceeds from the volume, utilizing a reference measurement of the IR light radiated onto the volume to be investigated that takes place by way of the optical reference path, and using a lock-in method, whereby the optical reference path has a measurement path characteristic that deviates from the measurement path characteristic of the optical measurement path.

In the present context, a value that is proportional to the concentration or inversely proportional to the concentration, for example, can be determined as a relevant value for the concentration. This determination can particularly serve for output of an analytical measurement value. Likewise, however, depending on the requirements, a digital output having a lower information content, for example to display a normal, a problematic and a critical concentration value or only a binary signal, for example when a critical concentration value is exceeded, can be determined as a “relevant value.” In this regard, the term “relevant value” in the present context refers to a value that serves for acquisition of the information content desired with the present determination of concentration, as a function of the concentration.

Up to the present, such determinations of concentration are relatively complicated. They require either a complex chemical analysis measurement structure or, the concentration is calculated using a spectral analysis. In this connection, it is understood that such measurement methods are extremely complicated if only because of their apparatus effort and expense, and, in particular, cannot easily be carried out by laypersons, on location. The same holds true for chemical measurement methods, which furthermore require a great consumption of aids, such as measurement strips or measurement chemicals, for example.

For example, WO 2005/112740 A2 and EP 0 670 143 B1 disclose measurement devices having a relatively simple structure, which can be operated by medical laypersons, if necessary, and particularly do not require any additional aids, such as measurement strips or measurement chemicals. However, WO 2005/112740 A2 does without a reference measurement, so that it is not of the same type if only for this reason.

DE 100 20 615 C2 on the other hand discloses, on the other hand shows a more complex measurement structure a more complex measurement device, which particularly requires a reference sample, in other words a measurement chemical, and in this way ensures that the optical paths between light source and measurement detector, on the one hand, and light source and reference detector, on the other hand, have identical measurement path characteristics, in each instance, so that in particular, sample and reference sample can also be interchanged. Such a measurement device, not of the type stated, is not suitable for measurements that are simple to carry out.

An array of the type stated is disclosed by EP 0 670 143 B1, in which a radiation source that is both amplitude-modulated and wavelength-modulated is used, whereby the amplitude modulation serves to vary the penetration depth of the light into a medium to be investigated, and the wavelength modulation serves to determine the first derivation of the spectrum at a selected wavelength, for which purpose a lock-in is also used. In the final analysis, however, a conclusion concerning the concentration has to be drawn from precisely one wavelength, and this is connected with significant uncertainty.

It is the task of the present invention to create a remedy in this regard.

As a solution, the invention proposes, on the one hand, a measurement device for optical determination of the concentration of blood sugar and/or lactate in biological systems, having at least one IR radiation source that radiates IR light onto a volume to be investigated, having at least one measurement detector that absorbs light proceeding from the volume to be investigated, and having at least one reference detector to which the IR light radiated onto the volume to be investigated is passed before entry into the volume, whereby the radiation source, the reference detector, and the measurement detector are connected with one another by means of a lock-in, and an optical measurement path is formed between the IR radiation source and the measurement detector, having a first measurement path characteristic, and an optical reference path is formed between the IR radiation source and the reference detector, having a second measurement path characteristic that deviates from the first measurement path characteristic, and whereby the measurement device is characterized in that the IR radiation source radiates IR light onto the volume to be investigated in at least two discrete wavelengths, i.e. in at least two discrete wavelength bands.

In this connection, it should be pointed out that purely physically, the photons of the light that are passed to the detector are not available for further measurement. In the present context, the term “light” accordingly refers to a bundle of beams or photons from which a part is, i.e. can be branched off for the reference measurement. In this regard, it is purely obvious that photons that are used for the reference measurement do not reach the volume to be investigated. Nevertheless, relevant statements concerning the nature of the IR light radiated onto the volume to be investigated can be made in this way, since these photons derive from one and the same radiation source.

Also, on the other hand, the invention proposes a method for optical determination of the concentration of blood sugar and/or lactate in biological systems, in which IR light is radiated onto a volume to be investigated, along an optical measurement path, and a value relevant to the concentration is determined from the light that proceeds from the volume, utilizing a reference measurement of the IR light radiated onto the volume to be investigated that takes place by way of the optical reference path, and using a lock-in method, whereby the optical reference path has a measurement path characteristic that deviates from the measurement path characteristic of the optical measurement path, and which is characterized in that at least one interesting peak from a previously determined or known spectrum is selected at least one component, and that the value relevant to the concentration is determined using at least two discrete wavelengths, i.e. wavelength bands that lie within this peak.

By means of the reference measurement, it is possible to determine variations in the IR light radiated onto the volume to be investigated, and in this manner to correct the value determination accordingly. Thus, it is possible to minimize possible temperature influences or also natural variations in the radiation source, and thereby to increase the accuracy of the relevant value with regard to the actual concentration. By means of such a measure, it is possible to significantly simplify the apparatus effort and expense, which are actually increased by the reference measurement in itself, in that lower requirements can be set for the radiation source, so that this source can be selected to be significantly smaller and easier to handle. In particular, temperature-stabilizing measures, i.e. other measures that appear necessary for stabilization of the radiation source, can be eliminated, and this makes it possible to significantly reduce the apparatus effort and expense as a whole.

A structurally simple embodiment can be implemented by means of a beam splitter that is disposed between the volume to be investigated and the radiation source. In this connection, the beam splitter is preferably oriented in such a manner that part of the light that proceeds from the radiation source is directed at the reference detector. In this manner, a reference measurement can be made in very simple structural manner.

Likewise, it can be advantageous if the beam splitter is oriented in such a manner that at least part of the light that proceeds from the volume is directed at the measurement detector. Such an embodiment can be selected, for example, to the effect that the volume to be investigated, such as a finger, an earlobe, or an arm, for example, is disposed between beam splitter and measurement detector. This applies particularly for the case that an absorption measurement of the IR light is being made. On the other hand, a reflection measurement and/or a measurement of light emitted some other way, which is stimulated by the exciting IR light, can be measured, particularly if this light proceeding from the volume reaches the beam splitter and is passed to the measurement detector proceeding from the beam splitter.

Accordingly, the measurement detector can be oriented in linear manner with regard to the IR light radiated onto the volume, for example, and this is particularly advantageous for absorption measurements. Particularly for reflection measurements, on the other hand, it can be particularly advantageous if the measurement detector is oriented at an angle with reference to the IR light radiated onto the volume. By means of the latter measure, it can particularly be avoided that the IR light radiated onto the volume passes directly through the volume and reaches the measurement detector without having been influenced.

A beam splitter is not absolutely required for the reference measurement. In an alternative embodiment, the reference detector can be directed at scattered light of the IR radiation source, for example. In the generation of light, independent of what light source is used, the formation of scattered light can hardly be avoided, since the light that is formed is already partially refracted in the radiation source itself, or otherwise slightly deflected. This is particularly true also for laser light sources. Because of the linear nature of photon propagation, laser light sources furthermore require two mirrors that stand opposite one another, between which the laser state can form. One of the mirrors is generally selected to be semi-permeable, so that the laser light can be uncoupled for further use by means of this mirror. However, other uncoupling mechanisms are also possible, but these in turn generate scattered light, which can be utilized for the reference measurement. However, since even an impermeable mirror generally allows a small proportion of light to pass through, i.e. can be configured to be slightly light-permeable, if necessary, it is easily possible, even when using a laser, to utilize scattered light, i.e. a small proportion of the light from the side that lies opposite the exit window of the laser for reference measurements. The use of such scattered light or outside light, respectively, for the reference measurement has the advantage that the main beam path between IR radiation source and volume is not hindered by the reference measurement. In this manner, losses can be minimized, and this in turn leads to simplification of the apparatus required for the desired measurement accuracy.

The lock-in method according to the invention, as well as a lock-in according to the invention, are sufficiently known from the state of the art, in themselves, in order to be able to eliminate statistical variations that influence measurement results. In this connection, an excitation signal that is directed at the sample is modulated, whereby it is assumed that a reaction of the sample induced by the modulated excitation is modulated accordingly, so that all the measurement signals that do not have a corresponding modulation are accordingly eliminated in the detector, and measurement signals that have the same modulation can be amplified. Depending on the concrete embodiment, both amplitude modulation and frequency modulation are possible for such modulation. However, such lock-in methods are not usual in spectroscopy studies, since such modulation leads to disturbances in the Fourier transformation. In the case of chemical determinations of concentration, as well, such lock-in methods cannot be used, of course.

However, very detailed statements concerning the light proceeding from the volume to be investigated can be made by means of the combination of an optical determination of concentration, i.e. the determination of concentration by way of IR light, with a lock-in method, and these statements lead to meaningful relevant values, at a relatively low apparatus effort and expense, despite everything.

Preferably, the reference detector is also connected with the IR radiation source by way of the lock-in, so that here, as well, interference effects, such as noise, for example, can be detected.

As already indicated above, an amplitude modulation can be performed for the lock-in method, which modulation can particularly be configured in binary manner, in other words “light on” and “light off.” In the case of an electronic embodiment of the lock-in, in particular, however, the amplitude modulation can be selected to be significantly less aggressive, and can move at low amplitude variations, for example in a window of below 50% of the maximal intensity, preferably of below 35% or below 25%, respectively. In particular, the amplitude modulation can also be structured as a sine wave, and thus less aggressively than the rectangular wave of a binary signal.

On the other hand, wavelength modulation can also be performed, whereby the bandwidth of the wavelength modulation preferably lies below 20 nm, preferably below 18 nm or below 15 nm, respectively, thereby making it possible to particularly ensure that a meaningful peak of the spectrum of the component whose concentration is supposed to be determined is not departed from as the result of the lock-in, i.e. as a result of the wavelength modulation. In this connection, it is understood that supplemental measures must be provided, if necessary, in order to be able to determine absolute values from a lock-in generated by way of wavelength modulation, since in this connection, the first derivation of a spectrum is generally measured first. On the other hand, specifically the first derivation of a spectrum can be just as meaningful with regard to a concentration as the spectrum itself, particularly if the wavelengths are suitably selected.

In the present case, laser diodes, in particular, can be used as radiation sources. At first glance, laser diodes appear unsuitable for spectroscopic studies with which determinations of concentration are usually carried out, since they do not have the required bandwidth for spectroscopic studies. On the other hand, such laser diodes have the advantage of a comparatively good light yield, so that excellent radiation power values, particularly also in the IR range, can be achieved with comparatively little apparatus effort and expense. Also, laser diodes can easily make light available for reference measurements, as was already explained in greater detail above with regard to laser light sources in general.

Because of the low modulation possibilities of the laser diodes, it can be advantageous to provide two or more laser diodes, and this can particularly be advantageous for time reasons, since modulation of a laser diode, particularly beyond a bandwidth of more than 20 nm, is relatively time-consuming, since the laser diode requires some time to stabilize. Furthermore, at such a bandwidth, the apparatus effort and expense increases disproportionately.

If at least one second peak is selected and one of the two wavelengths, i.e. one of the two wavelength bands lies in this second peak, the measurement accuracy can be increased, if necessary. Likewise, if multiple peaks are selected, multiple components, particularly of a biological system, can be checked with regard to their concentration.

Depending on the concrete implementation, it is particularly advantageous if different laser diodes, i.e. different IR light sources are used for measurements at the different peaks.

The relevant peaks, which are well accessible with IR measurements, generally have a width of far more than 200 nm. Because a wavelength band that lies within a peak, in each instance, is utilized, a profile of apparatus requirements is created that can be implemented with relative little effort and expense, particularly also by a laser diode. Thus, the effort and expense of modulating the wavelength of a laser diode, but also of any other IR radiation source, in a wavelength band below 200 nm, preferably below 170 nm, is disproportionately less than if multiple peaks would have to be detected—and thus multiple 100 nm made available as a bandwidth for the IR radiation source.

The present invention is particularly suitable for embodiments in which the wavelength bands, i.e. the emission frequency of the IR radiation source lies between 1000 nm and 2000 nm and/or between 2000 nm and 3000 nm. In this wavelength range, water, the main component of most biological systems, has a relatively slight interaction with light. In this way, components that can be found in such an environment are better accessible to measurement. It is understood that in the case of other environments, other wavelength ranges can also advantageously be used, if applicable.

In a concrete implementation of the method described here, it can be advantageous, on the one hand, to record only two measurement points at the selected peak, of which one measurement point lies in a range of the selected peak that is independent of concentration, while the other lies in a range of the peak that is as greatly dependent on concentration as possible. Then, a statement concerning the absolute concentration of the component in the volume to be investigated can be made from the incline between these two measurement points, whereby the measurement point in the range that is independent of concentration then serves as a reference. It is understood that the two measurement points, i.e. the two corresponding wavelength bands do not necessarily have to be found in the same peak.

In this connection, these two measurement points are controlled discretely, which can be implemented, on the one hand, by means of two laser diodes, for example, or, on the other hand, by means of a laser diode whose wavelength is accordingly changed suddenly, i.e. discretely. While it is true that laser diodes can be stabilized relatively precisely, to a few nanometers, this is relatively time-consuming, however, so that two laser diodes, in particular, are suitable for discrete control. On the other hand, the accuracy of the selection of the measurement points is not necessarily restricted to below one nanometer. Instead, it can be sufficient if the measurement points lie within a wavelength bandwidth below 20 nm. In this connection, it is particularly possible, as well, to modulate the frequency of the IR radiation source within the bandwidth of 20 nm, in order to utilize this modulation for a lock-in method. On the other hand, it can also be advantageous to stabilize the IR radiation sources to below 8 nm, preferably to below 6 nm or 4 nm, respectively, and to set them with corresponding precision, in order to carry out the lock-in at a low modulation or in order to implement an amplitude-modulated lock-in.

Depending on the IR radiation source used in a concrete case, however, it can be advantageous, on the other hand, to continuously modulate the IR radiation sources in a range below 170 nm, preferably below 150 nm or below 120 nm, respectively, and to determine the shape of the selected peak from this modulation, from which shape the value relevant for the concentration can be determined. A wavelength-modulated lock-in or wavelength-modulated lock-in, respectively can be implemented very well by means of such a large wavelength band.

It is true that the solutions explained individually above are advantageous in themselves, independent of the other characteristics of the other solutions. In practice, however, it has been shown that an apparatus structure can be created, in particular, by means of the combination of discrete wavelength measurements, reference measurements, and lock-in, while doing without a reference sample, which structure can be implemented to be small and guarantees sufficient measurement accuracy, and therefore allows measurements on location, particularly also by laypersons.

Elimination of the measurement of a complex spectrum, in particular, and instead, a restriction to one or more individual peaks during a determination of concentration, allows a measurement using laser diodes, which only have to be changed in their wavelength in restricted manner. If necessary a change in wavelength can be eliminated entirely, in that only two measurement points, i.e. narrow wavelength bands on an interesting peak are selected, and these are excited by means of a laser diode, in each instance. It is understood, in this connection, that more complex measurement procedures can also be carried out, without any significant loss of time, by increasing the number of laser diodes.

Independent of the asymmetry of the optical paths, in other words of the optical measurement path and the optical reference path, the use of multiple discrete wavelength bands, which are excited in wavelength-modulated manner, in each instance, is correspondingly advantageous in an optical determination of the concentration of blood sugar and/or lactate in biological systems, with at least one IR radiation source that radiates IR light onto a volume to be investigated, with at least one measurement detector that absorbs light proceeding from the volume to be investigated, and with at least one reference detector to which the IR light radiated onto the volume to be investigated is passed before entry into the volume, whereby the radiation source, the reference detector, and the measurement detector are connected with one another by means of a lock-in. The same holds true for a measurement method for optical determination of the concentration of blood sugar and/or lactate in biological systems, in which IR light is radiated onto a volume to be investigated, and a value relevant to the concentration is determined from the light that proceeds from the volume, utilizing a reference measurement of the IR light radiated onto the volume to be investigated, and using a lock-in method.

In the present context, the term “peak” refers to a significant variation of a spectrum that is characterized, in particular, by a maximum or a minimum, in other words an extreme point, and two foot points. In this connection, the foot points do not have to lie at the identical level, but are generally significantly less dependent on the concentration of the substance whose spectrum is being investigated than the extreme points, in each instance. In particular, as was already explained in detail above, it is possible to use multiple peaks, if necessary also only the extreme point of one peak and the foot point of another peak, for the measurements according to the invention.

Other advantages, aims, and tasks of the present invention will be clarified using the following description of the attached drawing, in which measurement devices and ways of conducting the method are shown as examples. In the drawing, the figures show:

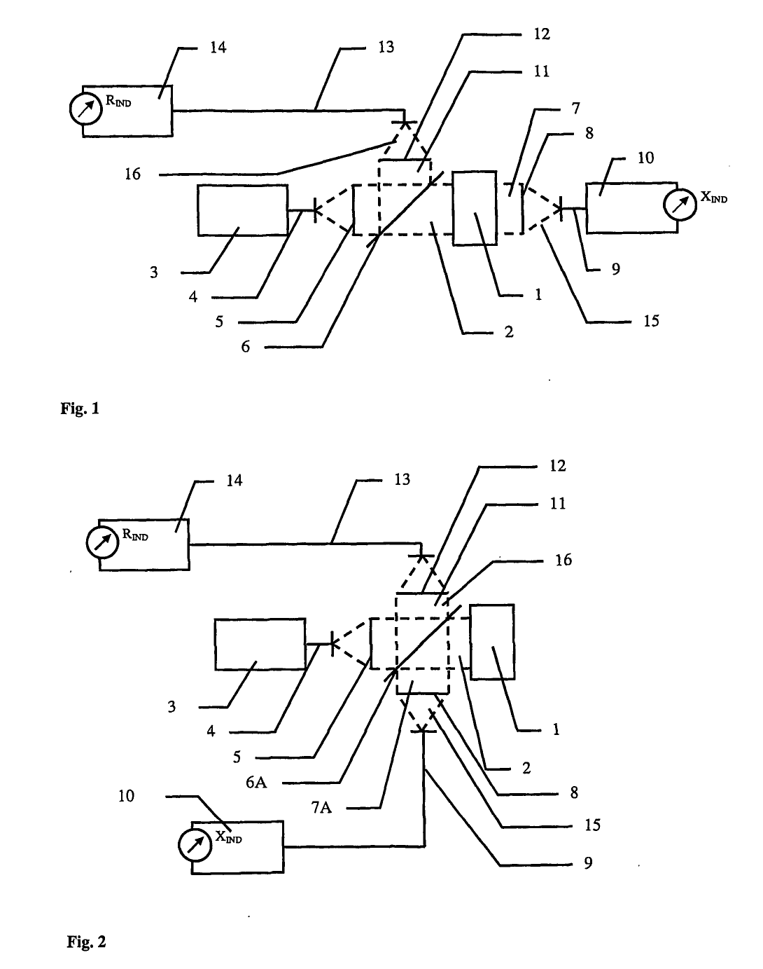

FIG. 1 a schematic structure of a first measurement array;

FIG. 2 a schematic structure of a second measurement array;

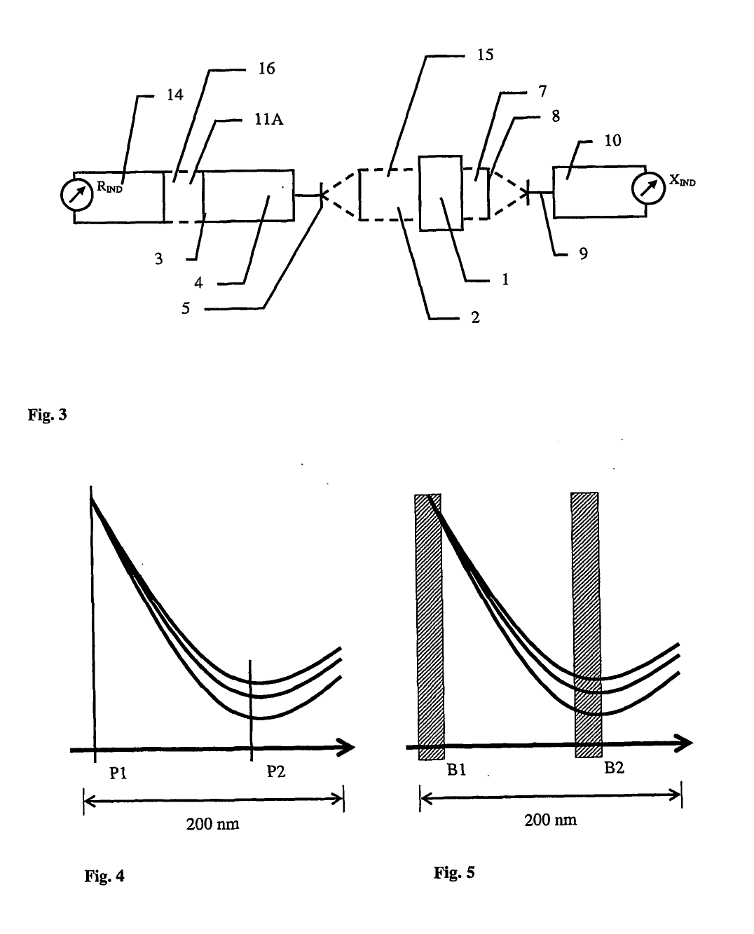

FIG. 3 a schematic structure of a third measurement array;

FIG. 4 a first possible way of conducting the method;

FIG. 5 a second possible way of conducting the method; and

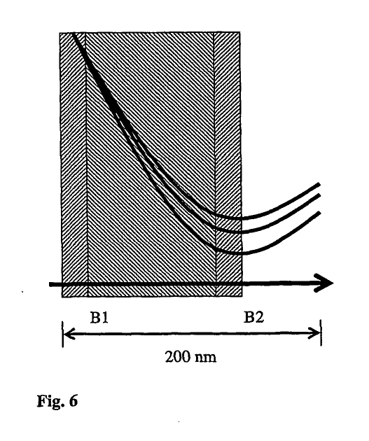

FIG. 6 a third possible way of conducting the method.

The measurement array according to FIG. 1 comprises a volume 1 in which the concentration of a component is to be determined. For this purpose, IR light 2 is radiated onto the volume 1, which light is generated by an IR laser diode 3 and passed to the volume 1 by way of a light guide fiber 4 and a collimator lens 5 as well as a beam splitter 6. The IR light 2 passes through the volume 1, whereby the light 7 that proceeds from the volume 1 is bundled accordingly, into a light guide fiber 9, by way of a collimator lens 8, and passed to a measurement detector 10.

Part of the IR light from the IR laser diode 3 is passed to a reference detector 14 by way of the beam splitter 6, as reference light 11, by way of a second collimator lens 12 and another light guide fiber 13. The light beam 2 as well as the detectors 10, 14 are connected with one another, in terms of measurement technology, by way of a lock-in connection, not shown.

In the array according to FIG. 2, which essentially corresponds to the measurement structure according to FIG. 1, so that identically effective modules are also numbered identically, somewhat deviating modules are marked with the letter A, and instead of an absorption measurement, a reflection measurement takes place. The light 7A proceeding from the volume 1 is guided onto the collimator lens 8 at the beam splitter 6A, which therefore is effective both for the IR light 2 radiated onto the volume 1 and for the light 7A that proceeds from the volume 1, and passed to the measurement detector 10 in the manner already described above. With this array, other light that is induced by the IR light 2 can also be easily detected. In this regard, this array is not restricted to reflection measurements. In this connection, it is understood that the detection array of collimator lens 8, light fiber 9, and detector 10 can be provided at a different location, in order to detect light that proceeds from the volume 1.

In deviation from the array according to FIG. 1, the array of FIG. 3 utilizes scattered light or light 11A that exits through the rear part of the IR laser diode 3, respectively, as reference light. For the remainder, this array corresponds to the configuration according to FIG. 1 and is designed for absorption measurement.

FIG. 4 shows a detail of a peak that is selected from the known spectrum of the component whose concentration is supposed to be determined. The detail comprises a wavelength range of about 200 nm. Corresponding to the above explanations, a measurement is carried out at two measurement points P1 and P2, which are spaced apart in their wavelength by about 150 nm. In this connection, the measurement point P1 is selected in such a manner that it lies in a region of the peak that is relatively independent of the concentration. In contrast, the measurement point P2 has been placed in a region that is as significant as possible with regard to the concentration, so that a conclusion concerning the concentration can be drawn from the difference between these two measurement points, whereby the measurement point P1 serves as a reference, so that outside influences, particularly the influences of other components of the biological system, can be excluded or minimized. By means of this measure, as well, the measurement structure can be implemented very simply, cumulatively or alternatively.

As is directly evident, such a measurement can be carried out using the measurement arrays presented above, in that the IR laser diode 3 is tuned with the corresponding wavelengths, in each instance. On the other hand, it is directly comprehensible to every person skilled in the art that instead of one IR laser diode 3, i.e. instead of one IR radiation source, two IR radiation sources that are tuned with the corresponding wavelength can also be used.

While in the case of this exemplary embodiment, the IR laser diode is tuned with relative precision, and preferably is kept constant, with variations below 1 nm, when the measurement points P1 and P2, respectively, are being recorded, in the case of the method example according to FIG. 5, the laser diode is modulated with a bandwidth of approximately 15 nm, in order to implement a lock-in method. The latter can be implemented when the method is carried out according to FIG. 4, by way of amplitude modulation. In this regard, wavelength bandwidths B1 and B2 are utilized in place of measurement points in the method example according to FIG. 5.

On the other hand, the wavelength of the IR radiation source can also be modulated over a greater range, for example approximately 150 nm, so that in particular, also the ranges B1 and B2, within which the measurement result is recorded, i.e. utilized for the determination of concentration, can be covered. From these measurements, as well, which will, however, generally be very time-consuming, relevant values concerning the concentration can be determined, whereby a lock-in can also be implemented by means of a suitable modulation. However, it is also possible, despite possible wavelength modulation with such a bandwidth, to carry out amplitude modulation and utilize it for the lock-in, while the modulation is merely used to cover a corresponding wavelength band. In this connection, it is particularly evident that other wavelength bands can easily be exploited, particularly also by means of the use of other IR radiation sources.

As is directly evident, in the case of the method examples according to FIGS. 4 and 5, the measurement points or wavelengths P1 and P2 or wavelength bandwidths B1 and B2, respectively, are spaced discretely apart from one another. The same holds true for the wavelength bandwidths B1 and B2 within which the measurement takes place in the exemplary embodiment according to FIG. 6.

As is directly evident, the characteristic of the path that the light takes from the IR laser diode 3 to the measurement detector 10 and that can be referred to as the optical measurement path 15 deviates, in the case of all the exemplary embodiments, from the characteristic of the path that the light takes from the IR laser diode 3 to the reference detector 14 and that can be referred to as the optical reference path 16, since the measurement path 15 comes into contact with a medium that is situated in the volume 1, while the optical reference path 16 is not affected by this.

### REFERENCE SYMBOL LIST

1 measurement volume
2 IR light
3 IR laser diode
4 light guide fiber
5 collimator lens
6 beam splitter
6A beam splitter
7 light proceeding from the measurement volume
7A light proceeding from the measurement volume
8 collimator lens
9 light guide fiber
10 measurement detector
11 reference light
11A light exiting through the rear part of the IR laser diode 3
12 collimator lens
13 light guide fiber
14 reference detector
15 optical measurement path
16 optical reference path

1. Measurement device for optical determination of the concentration of blood sugar and/or lactate in biological systems, having at least one IR radiation source that radiates IR light onto a volume to be investigated, having at least one measurement detector that absorbs light proceeding from the volume to be investigated, and having at least one reference detector to which the IR light radiated onto the volume to be investigated is passed before entry into the volume, whereby the radiation source, the reference detector, and the measurement detector are connected with one another by means of a lock-in, and an optical measurement path is formed between the IR radiation source and the measurement detector, having a first measurement path characteristic, and an optical reference path is formed between the IR radiation source and the reference detector, having a second measurement path characteristic that deviates from the first measurement path characteristic, wherein the IR radiation source radiates IR light onto the volume to be investigated in at least two discrete wavelengths, i.e. in at least two discrete wavelength bands.

2. Measurement device according to claim 1, wherein a beam splitter is disposed between the volume to be investigated and the radiation source, in such a manner that part of the light that proceeds from the radiation source is directed at the reference detector, and wherein at least part of the light that proceeds from the volume is directed at the measurement detector.

3. Measurement device according to claim 1, wherein the reference detector is directed at scattered light of the IR radiation source.

4. Measurement device according to claim 1, wherein the measurement detector is oriented in linear manner with regard to the IR light radiated onto the volume.

5. Measurement device according to claim 1, wherein the measurement detector is oriented at an angle with regard to the IR light radiated onto the volume.

6. Measurement device according to claim 1, wherein the radiation source is a laser diode.

7. Measurement device according to claim 6, wherein the laser diode has an emission frequency between 1,000 nm and 2,000 nm and/or between 2,000 nm and 3,000 nm.

8. Measurement device according to claim 6, further comprising means for modulation of the laser diode over a bandwidth below 170 nm, preferably below 20 nm.

9. Measurement device according to claim 8, further comprising two laser diodes.

10. Measurement device according to claim 1, further comprising means for modulation of the wavelength of the IR radiation source at least within a discrete wavelength band.

11. Measurement device according to claim 10, wherein the modulation means are connected to interact with a lock-in.

12. Method for optical determination of the concentration of blood sugar and/or lactate in biological systems, in which IR light is radiated onto a volume to be investigated, along an optical measurement path, and a value relevant to the concentration is determined from the light that proceeds from the volume, utilizing a reference measurement of the IR light radiated onto the volume to be investigated that takes place by way of the optical reference path, and using a lock-in method, whereby the optical reference path has a measurement path characteristic that deviates from the measurement path characteristic of the optical measurement path, wherein at least one interesting peak from a previously determined or known spectrum is selected for at least one component, and wherein the value relevant to the concentration is determined using at least two discrete wavelengths, i.e. wavelength bands that lie within this peak.

13. Method according to claim 12, wherein at least one second peak is selected and wherein one of the two wavelengths or one of the two wavelength bands, respectively, lies in this second peak.

14. Method according to claim 12, wherein the relevant value is determined from the absorption of the IR light that passes through the volume.

15. Method according to claim 12, wherein the relevant value is determined from the reflection of the IR light radiated onto the volume and/or from the light emission of the volume stimulated by the IR light radiated onto the volume.

16. Method according to claim 12, wherein amplitude modulation is performed for the lock-in method.

17. Method according to claim 12, wherein wavelength modulation is performed for the lock-in method.

18. Method according to claim 17, wherein the wavelength modulation has a bandwidth of below 20 nm, preferably below 18 nm or below 15 nm, respectively.

19. Method according to claim 12, wherein the wavelength bands lie between 1,000 nm and 2,000 nm and/or between 2,000 nm and 3,000 nm, in each instance.

20. Method according to claim 12, wherein the width of the wavelength band lies in a range below 170 nm, preferably below 150 nm or 120 nm, respectively.

21. Method according to claim 12, wherein the wavelength band is traversed by an essentially continuous modulation of the wavelength.

22. Method according to claim 12, wherein at least two discrete wavelength bandwidths, spaced apart from one another, are controlled within the wavelength band.

23. Method according to claim 22, wherein at least one wavelength band lies below 20 nm.

24. Method according to claim 23, wherein at least one wavelength band lies below 8 nm, preferably below 6 nm or below 4 nm, respectively.
